SEQUENCE LISTING (26 sequences)

SEQ ID NO: 1 - DNA, 1371 bp, Artificial
  Allatostatin C receptor sequence AlstCR
catattgctgtgtacgtggtgacgcaggtgttgtacgcgctcgtctgcgtggtggggctgctcggcaacaccctcgtcatctacgtcgtgctgcgctactccaagatgcagaccgtcaccaacatgtacatcgtgaacctcgccatcgctgacgaatgctttcttatcggcatcccattcctgatcacaacgatgtctcttcgtgcgtggccgtttggacgcttcatgtgtaaggcctacatgatatctaccggaatcaaccagttcaccagctctatattcttgtgtataatgagtgcggatcgatacatagccgtgtgtcatccgatagccgcgccgcgactgcggacgccattcgtctctagagtggtgtcagctgcagcatggacagcgtcagctttggtgatgacgcctatatttatgtacacgacactcataccaacaccccatggtctgtcgtgcaatatagtgtggcccgagcaagagttcaataaaggccagacgtccttcactctgtactcgttcgctctcggtttcgccgcaccactcacccttatcttcgtgttttactgtctcgtgattcgcaaactgaagactgtcggccctaagaacaaatctaaagagaaaaagcgatcacacagaaaagtaaccaaattagtgctgactgtcattgctgtgtacgtgctgtgctggctgccgtactgggcgttccaggtagctctcatttactctccaccgaacgagtgcgccagccagatcactatcaccgtttttctggtagccgcgtgcttcagttacagtaactctgccatgaacccaattctttacgcctttctttctgacaacttcaagaaaagtttcctaaaggcctgcacctgtgcagctgggaaagatgtgaatgcaactcttcatgtggagaacagtgtgataccgaggaagcggggaggtggcaccgcgctgctggaggaggacacgctgatcgtgtccatcgactagagtagtaagactgtcggacgggacgccgtcggccgcagagactcgagccctgtctctcggccgccgagcagcccacgggtccagttttgtaccggttcaagttttaaatattagttgtaaggtgtgcggtgtgtgagatatgtgatggagttgttctgtttgtgtataaagtcaaaattgtatggagtcggttcggcctagggttgtaacgatgaatttactaaaatgtaattttttatcgcatagcgactgtaaggttactgtttgtatactgtgtatttaaggtgtaaaaaattgtacatatcgtgtaaatatttgtgaacttgtcataatgtcacagttgtaataaaattacgttcgtgaaaaaaaaaaaaa

SEQ ID NO: 2 - protein, 331 aa, Artificial
  Protein sequence allatostatin receptor C
HIAVYVVTQVLYALVCVVGLLGNTLVIYVVLRYSKMQTVTNMYIVNLAIADECFLIGIPFLITTMSLRAWPFGRFMCKAYMISTGINQFTSSIFLCIMSADRYIAVCHPIAAPRLRTPFVSRVVSAAAWTASALVMTPIFMYTTLIPTPHGLSCNIVWPEQEFNKGQTSFTLYSFALGFAAPLTLIFVFYCLVIRKLKTVGPKNKSKEKKRSHRKVTKLVLTVIAVYVLCWLPYWAFQVALIYSPPNECASQITITVFLVAACFSYSNSAMNPILYAFLSDNFKKSFLKACTCAAGKDVNATLHVENSVIPRKRGGGTALLEEDTLIVSID

SEQ ID NO: 3 - DNA, 534 bp, Artificial
  Adipokinetic hormone receptor AKHR nucleotidic sequence
gacctgatgtgccgagtcatgatgttcaccaggaccttcggactgtacctctccagcttcgtgcttatctgtatagctgttgatagatactacgccattttaaagcctttgaacgtgacatgggaggcgcgcgtgcgcagagcgctgattatcgcgtgggtgtgcgccggcctcgccagcctgccgcagagcttcatattccacgtagaggagcatcctgatgttaaggggtactaccagtgcgtgtcgtacgggtcgttgccgacggagtgccacgagtttgcgtacttcttggtaaacatgatcctcatgtacgtcatccctctgctgtccacgctgtactgctcctcggcagctttgttggaaatcattcggagatctaacacttctaatgataagatgcgacggagcggcgtgggtatactcgggcgggcgcgggctcggacactcaagatgacagtcactatagttctagtcttttttacatgttggtcaccctactactgttattgtctgtggtattggatcgacaag

SEQ ID NO: 4 - protein, 178 aa, Artificial
  Protein sequence AKHR receptor
DLMCRVMMFTRTFGLYLSSFVLICIAVDRYYAILKPLNVTWEARVRRALIIAWVCAGLASLPQSFIFHVEEHPDVKGYYQCVSYGSLPTECHEFAYFLVNMILMYVIPLLSTLYCSSAALLEIIRRSNTSNDKMRRSGVGILGRARARTLKMTVTIVLVFFTCWSPYYCYCLWYWIDK

SEQ ID NO: 5 - DNA, 30 bp, Artificial
  AlstCR Forward primer
gaagtgtctttctctagctatatcggatgc

SEQ ID NO: 6 - DNA, 30 bp, Artificial
  AlstCR Reverse primer
atgcgcagcaatgcgtagcaatgcgctctt

SEQ ID NO: 7 - DNA, 24 bp, Artificial
  AKHR Forward primer
gacctgatgtgcacgacagtcatg

SEQ ID NO: 8 - DNA, 22 bp, Artificial
  AKHR Reverse primer
gtcagatccaagtaccacagag

SEQ ID NO: 9 - DNA, 1086 bp, Spodoptera littoralis
  CDS (1)..(1086): Allatostatin A receptor encoding sequence
atggcgtcgactgaagacgaatttaggagcatatgccttaacttaaccagtgaggaggctatgcttggaggatgtaactacagcagcgaatatgaaaatggagaactgttggagaaggtcgtctcgagggttgtgcccatcttctttggcttcataggcatcgttggattagttggcaatgctttggtcgtcttagtggtcgctgccaaccctggcatgcggtccactaccaacctgctcatcataaacttggctgtcgctgacttgctctttgtgatcttctgcgttccattcactgctaccgactatgtcatgccacgatggcccttcggagactggtggtgcaaggttgtccagtacttcattgtagtcactgctcatgcgtccgtctacactctagtactcatgtctctagacaggttcatggctgtagtgcatcccatagcttccatgtctatcagaacagagaagaacgcattacttgctatagcttgcatatgggtggtgatattaaccacagctattcccgtgggcatctgccacggggagcgggaatactcctacttccatagaaaccattcctcctgcgtgttccttgaggagcaaggatatagcaagctagggttccagatgtctttctttttgtcttcctatgtgattcccttagctttgattagtgtattgtatatgtgtatgctgacgaggttgtggaagagtgctccaggagggagggtgtctgcggagagtcggagggggaggaagaaggtgacgagaatggttgtggtcgtagtggtggtgttcgcagtgtgctggtgcccaatacagataatcctgttggtaaaggcgctggacaagtaccacatcacatacttcacagtcactgcgcagatagtgtcgcatgtactagcctacatgaacagctgcgtcaacccagtgctgtacgccttcctctctgagaacttcagggtcgccttcaggaaggtaatgtactgcccgcccccctacaatgacggcatgtcaggccggccccagcccaccaagacgactcgtacaggcaatgggaactcctgccatgacatcgtctga

SEQ ID NO: 10 - protein, 361 aa, Spodoptera littoralis
MASTEDEFRSICLNLTSEEAMLGGCNYSSEYENGELLEKVVSRVVPIFFGFIGIVGLVGNALVVLVVAANPGMRSTTNLLIINLAVADLLFVIFCVPFTATDYVMPRWPFGDWWCKVVQYFIVVTAHASVYTLVLMSLDRFMAVVHPIASMSIRTEKNALLAIACIWVVILTTAIPVGICHGEREYSYFHRNHSSCVFLEEQGYSKLGFQMSFFLSSYVIPLALISVLYMCMLTRLWKSAPGGRVSAESRRGRKKVTRMVVVVVVVFAVCWCPIQIILLVKALDKYHITYFTVTAQIVSHVLAYMNSCVNPVLYAFLSENFRVAFRKVMYCPPPYNDGMSGRPQPTKTTRTGNGNSCHDIV

SEQ ID NO: 11 - DNA, 1191 bp, Spodoptera littoralis
  CDS (1)..(1191): Diuretic hormone receptor encoding sequence
atggcggagaagtgcctggcgcagaacgaagggctggcggaggaggagttctgccccgcgtactacgacgggctgctgtgctgggacgcgacgccctggaacacgctggcggtgcagcgctgcttcagcgagttccacggcgtccagtacgatgacacacagaacgcatcgcggatgtgcttggacggcgtgtggcacaactactcaaactacgcgaactgcacagaacgcatgaccaacgtgtcgccgactgacgtcaccagcctcatctacctcgccggctactcgctcagcctcgccgtgctttcactcgccgtcttcgtcttcctttatttcaaggacctgcgatgtctaaggaacacgatacataccaacctcatgtcgacgtatattctgtctgcctgcagctggatcttaaacttagctttacaaaattggtccgatgaggcccaacaagatcaaacctcgtgtacgattctggtcatatgtatgcactacttttacctcactaatttcttcgggatgcttgtagaaggtttgtacctgtacatgctcgtggtggagacgttcaccgctgaaaatattaaactgaaggtgtacactaccataggatggggtgcgccggcggtgttcctaacaatatgggttatatgcaggtgcttcgtcaacatcgtgccttccaaccctgctgatgggcttgccctggcaggtgaggctaagatgtgtatttggatgtcggagcaccaggtggattggatccataaagctccagcactcgctgggctcgccctcaacctcttcttcctcattaggattatgtgggtattaatcacaaaactgcggtctgcaaacacgttggagacggagcagtatcggaaagcaacgaaggcacttctagtcctgatcccactgctgggcatcaccaacctgctggtgctgtgtgggcccaatgacgactcctggttcgcacacgccttcgactacacgcgagcactcatgctttcaacacagggtttcttggtggctttgttctactgcttcatgaatacagaagtacggcacgcaatcagataccacgtcgagaggtggaagactgggaggaccatcggtggaggaaggaggagaggagcttcctactccaaggactggtctcctcggtcacgaacagaaagcatacgactcacggtatga

SEQ ID NO: 12 - protein, 396 aa, Spodoptera littoralis
MAEKCLAQNEGLAEEEFCPAYYDGLLCWDATPWNTLAVQRCFSEFHGVQYDDTQNASRMCLDGVWHNYSNYANCTERMTNVSPTDVTSLIYLAGYSLSLAVLSLAVFVFLYFKDLRCLRNTIHTNLMSTYILSACSWILNLALQNWSDEAQQDQTSCTILVICMHYFYLTNFFGMLVEGLYLYMLVVETFTAENIKLKVYTTIGWGAPAVFLTIWVICRCFVNIVPSNPADGLALAGEAKMCIWMSEHQVDWIHKAPALAGLALNLFFLIRIMWVLITKLRSANTLETEQYRKATKALLVLIPLLGITNLLVLCGPNDDSWFAHAFDYTRALMLSTQGFLVALFYCFMNTEVRHAIRYHVERWKTGRTIGGGRRRGASYSKDWSPRSRTESIRLTV

SEQ ID NO: 13 - DNA, 2250 bp, Spodoptera littoralis
  CDS (1)..(2250): LGR1 receptor encoding sequence
atgtattggagattatgtatttgggctgttgcctttattttactggggagcagagaagcagcatgccactcgatgagtgagagtgtgtctgtggtgctgcaccagaccaacatcacacggctcacggcaaaggtcatccatgctgcgggctaccatttgcatcgagttgagcatttatcaataatggatgcaccaaaattggatcacatagtagtggacgatctgactaagatgcctaatctcaaatctctgtttataacgcaagctccgcgactacacagggtgtcgcccctccctccgctgcccgagctacgcacattcatcatcacaacctccggcttggtggaagtccccaacctgagccacgtgcacgacaagaagaataacaactcactcgcctatctgcaagctatagacctagaaggcaaccacataaagcgcctatcgccgcacgcattgcgcgtgcgcgctgatcaagtgtccttgaactacaacttgctagaagaagtgcctgcttttgcgttcaagaatgcgcagatttcaaaattgagtttcagaggaaacacgaaactaaaacatattgatgaaaatgccttcgcatcgaatttgctcctccgacaattagatttaagtaacaccgctattacctccctgcctacagcaggactacagaacttggaaacccttagaatagaaaagacaccttcattgaaatacatcccatcgatttacgaatttcagagtctccagaaagcgtacctgacgcatcatttccactgctgcgcgttccacttccccgagcgacacaacccaacgagacacaagctctatgaaacgcaaattgctataatgcagtcgaaatgtgcgagcgctagtcagcagaagaaaccagtagccagaagaaagaagaggtcactggcaccactcaagatcgttatgcctcagcgtaaaaacgatactacagactcattagaggaagacctgtcaacagcgactacgagcgaggagtacgacgctaacttgatggacgagtacttctcagactccggcagttgggaggacgaggacgaccagggagagttccacgaaccagtcaacaacacgctcggggccggcttcgcgatcatatccgctgagtgcgggaacttcagtattagcccacgaaaagtggaatgctttcccgcacctgatgcactgaatccgtgcgaagatgtgatgggctggtcgtggctccgagcgagtgtgtggttcgtcatcacggcggcggtggtgggcaacgttgccgtcctgctcgtcttacttacaaaccacacggagattacagtcccgcggttccttatgtgccacctcgccttctctgacctctgcaccggcctctacctgtttatgttggccgtcgtagacctcagatcttatggagaattcttcaactacgcctacgattggcagtacggggtcggctgcaagatcgctggctttctatcagtattttctggacagttatcagtaattactttaactatagtcacattggagcgctggttcgccataacttatgcaatatacttagaaaggaggatctctctatcagccgcagctaagataatgatgggcggttggttgtactccttcctgatggctggcttgccgctggtcggcgtgtcagactattcgtctacgtcaatatgtttaccggttgagagcaaggatgtgggagatgctgtgtaccagggatcaatatttatagtgagtgcgttggcgtgggtcacgattgtggtgtgctacgtgcagatatacaggtcgctagggggtggagagagttacggagggaggggggcggccgcagccgctgagagacgaattgcgaataaaatggcactcctcatcggaaccgacctgttgtgctgggcgcctgtggcgttcttcggcatcacagctctagcaggagtgccgctagtcgatgtcagtcacgggaaagtgttgctagtattcttttaccctctgaacgcttgtgcgaatccattcttatacgccatattaacgaaacaataccgaagagatttgataacgttgatagcacggacgggtcactgcacctggttagtggagaagtacaagttggcggcgaccccgccccccaccgcgcacactaacccttctgcgccggcgcagttgatgccgctagtagactccgtcacgtatcaaaggagccagtcacagtgtaaagacagtagtgaggtaccgctttaa

SEQ ID NO: 14 - protein, 749 aa, Spodoptera littoralis
MYWRLCIWAVAFILLGSREAACHSMSESVSVVLHQTNITRLTAKVIHAAGYHLHRVEHLSIMDAPKLDHIVVDDLTKMPNLKSLFITQAPRLHRVSPLPPLPELRTFIITTSGLVEVPNLSHVHDKKNNNSLAYLQAIDLEGNHIKRLSPHALRVRADQVSLNYNLLEEVPAFAFKNAQISKLSFRGNTKLKHIDENAFASNLLLRQLDLSNTAITSLPTAGLQNLETLRIEKTPSLKYIPSIYEFQSLQKAYLTHHFHCCAFHFPERHNPTRHKLYETQIAIMQSKCASASQQKKPVARRKKRSLAPLKIVMPQRKNDTTDSLEEDLSTATTSEEYDANLMDEYFSDSGSWEDEDDQGEFHEPVNNTLGAGFAIISAECGNFSISPRKVECFPAPDALNPCEDVMGWSWLRASVWFVITAAVVGNVAVLLVLLTNHTEITVPRFLMCHLAFSDLCTGLYLFMLAVVDLRSYGEFFNYAYDWQYGVGCKIAGFLSVFSGQLSVITLTIVTLERWFAITYAIYLERRISLSAAAKIMMGGWLYSFLMAGLPLVGVSDYSSTSICLPVESKDVGDAVYQGSIFIVSALAWVTIVVCYVQIYRSLGGGESYGGRGAAAAAERRIANKMALLIGTDLLCWAPVAFFGITALAGVPLVDVSHGKVLLVFFYPLNACANPFLYAILTKQYRRDLITLIARTGHCTWLVEKYKLAATPPPTAHTNPSAPAQLMPLVDSVTYQRSQSQCKDSSEVPL

SEQ ID NO: 15 - DNA, 1437 bp, Spodoptera littoralis
  CDS (1)..(1437): OCt/Tyr receptor encoding sequence
atggggcaaacagctacacacgtaaacgcaaattacacattaattaattatactgatgaaataattgaggatgacagagacgcttgtgccgtcgcagatgaccctaaatatccgagtagtttcggtataacacttgcggtaccagaatgggaagcaatttgtacagccatagtcctaacgcttataataatctcaacgatagttggaaacattttggtgatcttgagtgtgtttacatacaaaccccttaggattgtccagaacttcttcatagtatcacttgctgtggcggatttgacagtagcaatattagtattgccactaaatgtggcatactccattctaggacaatgggtatttgggatttacgtatgcaaaatgtggttaacatgcgacataatgtgttgcacttcgtccattttaaatttatgtgcaatcgcattagacaggtattgggcgataaccgatcctataaattatgcacaaaagagaactttagaaagagtgatgttaatgattggagtagtttgggtactttcattagttattagctctccgccacttctagggtggaatgactggcccgacgtctttgaacctgatacgccttgtcgtttaacttcccaacctggcttcgttatcttctcttcatctgggtccttctacataccgctagttataatgactgtagtttattttgaaatatatttggccactaaaaaaagacttagagatcgcgctaaagccacaaagatcagcactatttctagcggtcagaatagatacaataataaagacgataacaatgatcatgatcaagattctgttagctcagaagccaatcacaatgagcaccaaggcgtgacacggttggcatcagacaatgagaaaaagaaaagaaccagaaaattgacaccaaagaaaaaaccaaagcgacgatattggagtaaggacgataaatcgcaaaacaaactgataataccgattctctctaacgataattcggtaactgatatgggtgataatttagaaaatagaaacacatcatcagagagtaactcgaaagaaactcacgaggatgatttgattgaagttactgagcctgcgccagtgaagagtcacaagcggccgaagcctaaccagcaaagtgctgtttatcaattcattgaagaaaagcaaaggatttccttgacacgtgagcgtcgagccgccagaacccttgggattataatgggcgtatttgtcgtgtgttggttgcccttctttgtcatataccttgttattcccttctgtgcgagttgctgtttgtccaacaagtttatcaatttcataacttggcttggctactgcaattctgcactcaaccctctaatatacaccatctttaacatggactttaggagagcattcaaaaagttactctgtatgaaaccgtga

SEQ ID NO: 16 - protein, 478 aa, Spodoptera littoralis
MGQTATHVNANYTLINYTDEIIEDDRDACAVADDPKYPSSFGITLAVPEWEAICTAIVLTLIIISTIVGNILVILSVFTYKPLRIVQNFFIVSLAVADLTVAILVLPLNVAYSILGQWVFGIYVCKMWLTCDIMCCTSSILNLCAIALDRYWAITDPINYAQKRTLERVMLMIGVVWVLSLVISSPPLLGWNDWPDVFEPDTPCRLTSQPGFVIFSSSGSFYIPLVIMTVVYFEIYLATKKRLRDRAKATKISTISSGQNRYNNKDDNNDHDQDSVSSEANHNEHQGVTRLASDNEKKKRTRKLTPKKKPKRRYWSKDDKSQNKLIIPILSNDNSVTDMGDNLENRNTSSESNSKETHEDDLIEVTEPAPVKSHKRPKPNQQSAVYQFIEEKQRISLTRERRAARTLGIIMGVFVVCWLPFFVIYLVIPFCASCCLSNKFINFITWLGYCNSALNPLIYTIFNMDFRRAFKKLLCMKP

SEQ ID NO: 17 - DNA, 1419 bp, Spodoptera littoralis
  CDS (1)..(1419): G protein coupled receptor SlOR83b encoding sequence
atgaccaaagtgaaggcccagggcctcgtctccgacctgatgcccaacatcaagttgatgcaggcagctggacacttcctcttcaattaccatgcagaaaatggtggcatgagcggcctccttcggaagatctacgccagtacccacgcgattctcatcaccatacattttgcctgtatgggtatcaacatggcccagtactccgatgaggtcaacgagctgactgctaacactatcaccgtcctcttcttcactcacaccatcatcaagttggggttcttcgctttgaactccaagagtttttacaggactttagcagtatggaaccagtcgaacagccaccctctgttcacggagtcagatgcccgctaccatcaaattgcgctcaccaagatgaggagactcctgtacttcatctgcggcatgactgtcctgtcagttgttagctgggtcacactcacattcttcggtgaatcagtccgcctgattactagtaaggaaacgaatgagacgttgacagaagtagcccctcgattgcctctgaaggcctggtaccctttcaatgctatgagcggcaccacgtatattatcgctttcgctttccaggtctactggctcctcttctcaatggctatagcaaatctgatggatgttatgttctgctcctggctgatcttcgcttgtgaacagctgcagcacctgaaggccatcatgaaacctctcatggaactcagtgcttcactggacacgtatcggcctaatactgctgagctgttccgtgcttcttccactgagaaatcagaaaagattcctgatacagtcgacatggacatccgcggcatatattccacacagcaagatttcgggatgacgttgcgaggagctggaggtagactgcaggcctttggacagcagaacaacaaccctaatggcttgactccgaagcaggagatgctggcgaggtctgctattaagtattgggtggagaggcacaaacacgtcgtcagattggtagcatcgataggagacacgtatggtactgccctgctgttccacatgttggtgtcaactataacactcactctattggcttaccaggctactaagatcaatggaattaatgtctacgcgttcagtactattggctacctcagctataccctgggacaggtgttccatttctgtatcttcgggaacaggcttattgaagagagttcctcagtgatggaagccgcctactcctgtcagtggtacgacggctccgaggaagcaaagaccttcgtccagatcgtgtgccagcaatgccagaaagccatgagcatctccggggccaagttcttcacggtgtcacttgatctttttgcttcggtacttggtgcagtagttacttacttcatggtgttggtacaactcaagtaa

SEQ ID NO: 18 - protein, 472 aa, Spodoptera littoralis
MTKVKAQGLVSDLMPNIKLMQAAGHFLFNYHAENGGMSGLLRKIYASTHAILITIHFACMGINMAQYSDEVNELTANTITVLFFTHTIIKLGFFALNSKSFYRTLAVWNQSNSHPLFTESDARYHQIALTKMRRLLYFICGMTVLSVVSWVTLTFFGESVRLITSKETNETLTEVAPRLPLKAWYPFNAMSGTTYIIAFAFQVYWLLFSMAIANLMDVMFCSWLIFACEQLQHLKAIMKPLMELSASLDTYRPNTAELFRASSTEKSEKIPDTVDMDIRGIYSTQQDFGMTLRGAGGRLQAFGQQNNNPNGLTPKQEMLARSAIKYWVERHKHVVRLVASIGDTYGTALLFHMLVSTITLTLLAYQATKINGINVYAFSTIGYLSYTLGQVFHFCIFGNRLIEESSSVMEAAYSCQWYDGSEEAKTFVQIVCQQCQKAMSISGAKFFTVSLDLFASVLGAVVTYFMVLVQLK

SEQ ID NO: 19 - DNA, 1053 bp, Spodoptera littoralis
  CDS (1)..(1053): PBAN receptor encoding sequence
atgacattgtcagcgcccccgatcgacgagtttgaggacccgttcgtcgtgatgaacacgacgaatgtgtcgagtcacccggccgcttatgacgagccatacacgctcgatctggtggtgccactcaccgtcacctacgtggtgatattcgtagccggcatcctggggaacaccagcacctgtgtggtcattgcgaggaaccgctctatgcacaccgctacaaacttttacctcttcagtttagcaatttcggatctcatattgttagtatgtggtctgccgtttgaggtgcacagattatggaacccggacacgtacccgttgggcgaggctcactgcatcgccattgggttagcgtcggagacttcagctaacgcgacagtgttgacaataacagcgtttacggtggagcggtacattgcgatatgtagacctttcatgtcgcacaccatgtcgaagctgtcacgggctgtgcggtttataatagctatttgggtgtttgcgttgtgtactgcggtaccacaagcgatgcagttcggtatagtgtcgtatgtggataatggtcagaatgtgagcgcgtgcacagtgaagggtgtgggtgttcatcaagtgttcgttatatctagtttcgtgttctttgtggtgcctatgtcgatgatatcagtgttgtacgcgttgatcggcattaagttgcggacctcacgagtgttgcatccagtgaagaagctctcggtggacagtaacgaaagagcgagtggacagatgcagtacaggaatggagcatcacagaggagagtcattagaatgctggttgcggtggcgctgtcattcttcatttgctgggcgcccttccacgtgcaaaggttgctggccatctacgggaaaagtttagaacacccttctgatactttctacttggtctacatcgtgctgacgtttttgtcgggagtgctatatttcctgtcaaccgccatcaacccgttcctgtacaacatcatgtcgaataagtttagaaacgccttcaaggtgagtttttgttacattcatgattga

SEQ ID NO: 20 - protein, 350 aa, Spodoptera littoralis
MTLSAPPIDEFEDPFVVMNTTNVSSHPAAYDEPYTLDLVVPLTVTYVVIFVAGILGNTSTCVVIARNRSMHTATNFYLFSLAISDLILLVCGLPFEVHRLWNPDTYPLGEAHCIAIGLASETSANATVLTITAFTVERYIAICRPFMSHTMSKLSRAVRFIIAIWVFALCTAVPQAMQFGIVSYVDNGQNVSACTVKGVGVHQVFVISSFVFFVVPMSMISVLYALIGIKLRTSRVLHPVKKLSVDSNERASGQMQYRNGASQRRVIRMLVAVALSFFICWAPFHVQRLLAIYGKSLEHPSDTFYLVYIVLTFLSGVLYFLSTAINPFLYNIMSNKFRNAFKVSFCYIHD

SEQ ID NO: 21 - DNA, 944 bp, Musca domestica
  misc_feature (1)..(944): Nucleotidic sequence of the Octopamine receptor of Musca domestica
tgggcgataaccgatccaataaactatgctcaaaagcggacagttggcagagttttgctgttaatagctggtgtctggattttatcgctactcatcagttcgccaccgttggttggttggaatgattggccggaagagtttaccagtgccactccttgcgaactaaattctgatcctggctacgtaatttattcttcactgggatcattcttcattccattaatcataatgacattggtttatatagaaatatttgttgctacccggagacgactacgagaaagggcgcaggcggcaaagataaacacaattgccacacgatctgtcagcgctgatgtagcaagcactactgtgacggcaaatcaaacgggaaatggcaccatatccattttgtgttacagcgtcatttgttgctgccgcaactcgacgccattctctacaacgccgatgatacaaaacgaccaagagtccgaaagtagcgaaacaaatgccaacaacgaaagtacaagagttcaaaatcaaacagataccaagacaactgtggcttcacagcgaaaaaggacccgaagagccaaaatcaaagactccattaaacatggaaaactaaaggctcggtcaggtcatacatttaacgaccacccagaagtacagaaatctaagcagataggaagtggtgcggaaagtcttctactggcacccaatgccaaaggcacagaaaatgaaaatcaagaaatatctactgagagcggcagcgacgcaaaaggttgcatgcaagtgtgtggaagtcggaacgacgccaatgaaatctcagccggtgacggcgaggaagaaaatgtgtctctgaaaattaccccaccccagtcgtctgtaggcgcaacgtcgttgcaaacagttccattgcaaaagaaaccggggggcgtatatcagttcatcgaagaaaaaca

SEQ ID NO: 22 - protein, 314 aa, Musca domestica
  misc_feature (1)..(314): Protein sequence of the Octopamine receptor if Musca domestica
WAITDPINYAQKRTVGRVLLLIAGVWILSLLISSPPLVGWNDWPEEFTSATPCELNSDPGYVIYSSLGSFFIPLIIMTLVYIEIFVATRRRLRERAQAAKINTIATRSVSADVASTTVTANQTGNGTISILCYSVICCCRNSTPFSTTPMIQNDQESESSETNANNESTRVQNQTDTKTTVASQRKRTRRAKIKDSIKHGKLKARSGHTFNDHPEVQKSKQIGSGAESLLLAPNAKGTENENQEISTESGSDAKGCMQVCGSRNDANEISAGDGEEENVSLKITPPQSSVGATSLQTVPLQKKPGGVYQFIEEK

SEQ ID NO: 23 - DNA, 1338 bp, Leptinotarsa decemlineata
  misc_feature (1)..(1338): Octopamine receptor Leptinotarsa decemlineata encoding sequence
atgggagaagaacacaacaattgttccgatgagccgtacggcaatttctccaccatattcggttcaacagacattcctgaatgggaagtgatcatcacaataggaactttgtcccttttcatcctactcacagtaataggaaacgtcctcgtgattttgagcgtttttacctacaagccattgcgaatagttcagaattttttcatcatctctcttgctgtggccgacctgaccgtggctctccttgtccttccatttaatgtggcgtataccgttttagggcgatggatatttggtatccatgtctgcaaaatgtggctcacatcagacgtcttgtgctgcactgcctccattttgaatctttgtgcaattgctctcgacaggttttgggctataacagatcctataaactatgctcaaaaaaggacagttaaacgagtactggttatgatagcaggcgtatgggtgctatcacttttgatcagttcacctcctttgattgggtggaatgactggccatcatctgatgtattcgcagaaagacaaatttgtatgctcacaaaacgccaaggttatgtgatttattcatcttctggttccttttttatacctctattcatcatgacaatagtttatgtagaaatattcattgcgacaaaacgaaggctgagagaaaggtcccaagcagcgaaaattaacatatctaaaaatatgagtaaaattaaaaacaaagacgatcatcgcgatggtgaaagcgtcagtagcgaaaccaatcataacgaattcgaagcgaatggtaaatctagagataaatctttgagaaaagacaaatcgagcgaatctgataaaaatactcttcaaccgatattgactcatgaagattctctaactgattttggagacaactcatcctgctctcaaaggaaagactcgaatccctcaaaatgtgagaataagggaatttgtagttcaaccactaccataacgatctccaactcgaaacctagtccaaaagtaatactcagtgatcccaataaaaaacctggatttgtatatcagttcatagaggaaaaacagagaatatcactttctaaagagagacgagcagcaagaacgcttggtatcattatgggtgtttttgtcgtatgctggttaccctttttccttatgtacgtgataattccgttctgtccgtcatgttgtccttcaagagcattaacgaatgttatcacttggttgggttatatcaactcaggactcaatcccgtcatatataccattttcaatatggacttcagaagggctttcagaaaactgttaggtctttctcaaacctaa

SEQ ID NO: 24 - protein, 445 aa, Leptinotarsa decemlineata
  misc_feature (1)..(445): Octopamine receptor Leptinotarsa decemlineata protein sequence
MGEEHNNCSDEPYGNFSTIFGSTDIPEWEVIITIGTLSLFILLTVIGNVLVILSVFTYKPLRIVQNFFIISLAVADLTVALLVLPFNVAYTVLGRWIFGIHVCKMWLTSDVLCCTASILNLCAIALDRFWAITDPINYAQKRTVKRVLVMIAGVWVLSLLISSPPLIGWNDWPSSDVFAERQICMLTKRQGYVIYSSSGSFFIPLFIMTIVYVEIFIATKRRLRERSQAAKINISKNMSKIKNKDDHRDGESVSSETNHNEFEANGKSRDKSLRKDKSSESDKNTLQPILTHEDSLTDFGDNSSCSQRKDSNPSKCENKGICSSTTTITISNSKPSPKVILSDPNKKPGFVYQFIEEKQRISLSKERRAARTLGIIMGVFVVCWLPFFLMYVIIPFCPSCCPSRALTNVITWLGYINSGLNPVIYTIFNMDFRRAFRKLLGLSQT

SEQ ID NO: 25 - DNA, 39 bp, Artificial
  MdOctR Forward Primer
tgggcatcgatatcacatcggatcccatcgatatcaatc

SEQ ID NO: 26 - DNA, 38 bp, Artificial
  MdOctR Forward Primer
caatcgacatcgacagaaatcgacatcgcccatatgat

(19) United States
(12) Patent Application Publication
(10) Pub. No.: US 2013/0011372 A1
(43) Pub. Date: Jan. 10, 2013

(54) INACTIVATED MICROORGANISMS CONTAINING DOUBLE -STRAND RNA MOLECULES (DSRNA), THEIR USE AS PESTICIDES AND METHODS FOR THEIR PREPARATION

(75) Inventors: Assunta Tortora, Pagani (SA) (IT); Maria Rosaria Grimaldi, Quarto (NA) (IT); Alessandra Ruggiero, Napoli (IT); Lucio Filippini, Novara (IT); Fabio Apone, Napoli (IT); Maria Gabriella Colucci, Pozzuoli (IT)

(73) Assignee: ARTERRA BIOSCIENCE S.R.L, Napoli (IT)

(21) Appl. No.: 13/498,077

(22) PCT Filed: Sep. 17, 2010
(86) PCT No.: PCT/IB10/02363

§ 371 Date: Aug. 13, 2012

(30) Foreign Application Priority Data
Sep. 25, 2009 (IT) MI2009A001649

(51) Int. Cl. A01N 63/02; C12N 1/19; C12N 1/15; C12N 1/11; C12N 5/10; C12N 15/81; C12N 15/82; C12N 15/63; A01N 65/00; A01P 7/04; A01P 7/02; A01P 9/00; C12N 1/21; C12N 15/85

(52) U.S. Cl. 424/93.21; 435/252.3; 435/254.2; 435/254.11; 435/258.1; 435/348; 435/325; 435/419; 435/252.33; 435/254.21; 435/254.23; 435/455; 435/468; 435/476; 435/483; 424/93.2

(57) ABSTRACT

The present invention relates to a new group of pesticides consisting of inactivated microorganisms containing double-strand RNA molecules (dsRNA), corresponding to receptor genes coupled with G proteins (GPCR) whose functioning is vital for phytophagous invertebrates (insects, mites and molluscs) or for infesting or in any case harmful organisms for the health of human beings and domestic animals, and to a method for the preparation of said microorganisms.

The present invention relates to a new group of pesticides consisting of inactivated microorganisms containing double-strand RNA molecules (dsRNA) corresponding to receptor genes coupled with G proteins (GPCR) whose functioning is vital for phytophagous invertebrates (insects, mites and molluscs) or for infesting or in any case harmful organisms for the health of human beings, and to a method for the preparation of said microorganisms.

The discovery of RNA interference (RNAi) as a powerful and simple gene silencing method has aroused the attention of the whole scientific community. The ubiquity of the phenomenon has allowed it to be studied in many species, as the process has been preserved during its evolution.

This is a gene silencing mechanism whereby various double-strand RNA fragments (dsRNA) are capable of interfering and extinguishing the gene expression (Brantl S., 2002). Through an enzyme called dicer, the dsRNA sequence is cut into fragments having a shorter length (19-21 pairs of bases) (Hamilton and Baulcombe, 1999). The short dsRNA fragment (called short interfering RNA, or siRNA) is associated with an enzymatic complex called RISC(RNA-interference silencing complex). The dsRNA is opened, probably by a helicase: only the antisense RNA strand remains associated with RISC, whereas the sense strand is degraded. The RISC complex is capable of recognizing among the many mRNAs present in the cytosol, that which is complementary to the antisense RNA fragment associated with the same complex. If the pairing between siRNA and mRNA is perfect (or almost perfect), a component of RISC (called argonaute protein) is capable of effecting a cut on the mRNA (Hammond et al., 2001). The two resulting mRNA fragments, one hood-free at 5′ and the other poly-A tail-free at 3′, are thus rapidly degraded by the RNAsi of the same cell.

Another common protein, although not universally present in the RNAi machinery, is RNA-dependant RNA polymerase (RdRP) which synthesizes dsRNA starting from single-helix RNA pattern to create the amplification mechanism of RNAi.

Thanks to experiments on C. elegans, numerous genes involved in RNAi have been identified, of which the homologues in Drosophila, plants and fungi, were then identified. These studies have demonstrated that the phenomena which were first classified as PTGS (post-transcriptional gene silencing), quelling or co-suppression, are all part of a single process whose roots are embedded in a single ancestral mechanism. Silencing experiments were carried out using dsRNA on pathogens and phytophagous organisms of plants of agrarian interest to inhibit the expression of some of their vital genes. Micro-injections of dsRNA into the final stage larvae and adults of Tribolium castaneum beetles allowed genes to be silenced together with a study of their function (Tomoyasu and Denell, 2004). Various researchers performed assays with larvae of Diabrotica virgifera virgifera LeConte fed with a synthetic diet enriched with specific dsRNA to indentify essential genes for the vitality of the insect (Baum et al., 2007). These assays led to the identification of 14 essential genes whose inactivation due to interference, causes larval death. The same authors have demonstrated that transgenic corn plants expressing one of these dsRNAs show high protection levels, comparable to those obtained with transgenic plants expressing Bt toxin.

For the control of pathogen agents and phytophagous organisms, the selection of the gene target to be inactivated is of fundamental importance. For this purpose, the receptors coupled with G proteins (GPCR), a vast group of proteins structurally and functionally similar to each other, represent excellent targets as they exert functions of vital importance in all eukaryotic organisms (Hill, 2006). They are composed of a single polypeptide chain which crosses the membrane seven times having the amino-terminal end extracellular and the carboxy-terminal end intracellular; after binding with extracellular ligands (peptides, small molecules, ions, light and aromatic compounds), the receptor is activated by triggering a response in the cell which is translated into the production of a second messenger (cAMP, Ca²⁺, cGMP, IP₃).

Most insects of agrarian interest have numerous GPCRs, the majority of which are fundamental for the correct running of their vital functions. The most widely-studied species, whose genome has been completely sequenced and for which there is considerable information on the development biology, are the dipterans Drosophila melanogaster, Anopheles gambiae and the lepidopter Bombyx mori. Drosophila melanogaster is certainly the most widely-studied arthropod and is considered the model organism for the study of harmful insects. Approximately 270 GPCRs have been identified in the genome of Drosophila, grouped into 5 families.

As far as molluscs are concerned, there is no information on GPCRs of species harmful for agriculture, such as Helix spp., Cepacea spp., Arion spp. and Deroceras spp. Information is available in data banks on GPCR receptors of more widely-studied species. Aplysia californica, for example, represents a model species for the study of the nervous system and electrophysiology in molluscs, and parts of the genome of this species have therefore been sequenced. Other GPCR sequences have been identified in species of the kinds Lymnea, Crassostrea, Haliotis, Octopus e Loligo.

In conclusion, interference by RNAi on the GPCRs of species of insects or other harmful organisms appears to be extremely specific, as the gene homology percentage between the GPCRs of invertebrates and those of mammals never exceeds 20-30%. The percentage relating to the homology between the GPCRs of species belonging to the same order is also never complete (in insects for example, it is around 90%) and this guarantees discrimination between species of harmful organisms and those beneficial for the environment.

Most of the substances used as insecticides are currently molecules within a wide range, there is consequently an enormous demand for more selective methods which specifically interfere with the survival and fertility of harmful organisms alone, but which, at the same time, have a minimum or zero effect on other non-target organisms and on the environment.

The massive use of halogenated hydrocarbons, carbamates, pyrethroids and organo-phosphates against phytophagous insects, for example, has caused various toxic effects on the environment and on other non-target organisms, and has also generated resistance forms which have subsequently thwarted use against various species. Insecticides such as pyrethrins and pyrethroids (cypermethrin, tetra- and deltamethrin, bifenthrin), acetamidines (acetamiprid), organo-phosphates and nicotinoids, are also used against insects infesting household environments (flies, mosquitoes, ants and cockroaches). Although most of these products are used limitedly, they can cause toxicity, in both human beings and pets, and also in the environment. Pyrethroids, for example, can cause convulsions and vertigo, as far as serious nervous disturbances and a reduction in fertility. Cases are known of human contacts with organo-phosphate products which have caused stinging of the mucous membranes, breathing difficulties and abdominal cramp. Furthermore, nicotinoids are extremely toxic for aquatic organisms and are causing significant reductions in the populations of bee farms.

On the basis of what is specified above, the necessity is evident for availing of new methods and relative biological targets which allow specific interference with the survival and fertility of organisms harmful for plants and which infest household environments, which are more selective and which consequently have a lower environmental impact.

The authors of the present invention have prepared new pesticides and relative treatment and protection methods of plants, food products, human households, based on the use of deactivated microorganisms (bacteria, yeasts, fungi, protozoans or cells of higher organisms) containing in their interior molecules of dsRNA capable of inhibiting the functionality of a GPCR receptor which, once ingested by the phytophagous or infesting organism, causes its death or a reduction in its vitality. GPCR receptors can in fact be convenient targets for controlling harmful and infesting organisms as they have a gene sequence which is very specific for harmful organisms. The dsRNA molecules are consequently capable of inhibiting their functionality and cannot cause damage either to the plant treated or to non-target consumers of the same plant (i.e. human beings, other animals). These dsRNA molecules capable of inhibiting the functionality of a GPCR receptor are produced by specific microorganisms which, once deactivated, represent excellent vehicles for transporting the dsRNA as far as the digestive system of the harmful organism. The involucre, consisting of the dead microorganism (therefore no longer able to grow or reproduce itself) protects the dsRNA molecules from degradation, until they are ingested by the phytophagous insect, which feeds on plant tissues, or by an infesting organism which feeds on food products or scraps. The dsRNA molecules only exert their insecticide action when they penetrate inside the body of the specific insect against which they have been produced. The mortal effect or reduction in vitality is not exerted against other non-target organisms, even if they feed on the plant or accidentally ingest the microorganisms containing dsRNA. Each product contains specific dsRNA molecules for the target organism against which they are to act. One product however can also be effective against various species contemporaneously if it derives from the mixture of various preparations of microorganisms/dsRNAs, each one selective for a single species. This allows a significant control of the action and efficacy of the product, as it is strictly oriented towards the species to be struck, without interfering with any other non-target organism.

Once the product (i.e. deactivated microorganism containing dsRNA against GPCRs of interest) has been prepared, it can be dissolved in water (or in another water-based buffer solution) and sprayed onto the leaves and stem of vegetables (action against insects and other harmful phytophages), or mixed with appropriate baits (for insects infesting households, food products) or mixed with the excrements of farm animals (against insects infesting the environments of domestic animals), or sprayed on furniture or wooden structures (for xylophage insects), or administered in the environment, on the soil or in water ponds (against mosquito larvas).

In addition to being applied to agriculture, the invention is also applied for fighting all species of insects and mites which infest and damage human houses or domestic animals. Many species of insects in fact damage food products or crops stored before being transported and processed, infest human households creating the risk of transmitting diseases (flies, cockroaches, ants), or are a direct threat to architectural structures and objects of household use (termites, woodworm, moths). Furthermore, the technology described herein can also be used against all bothersome insects which, by molesting domestic animals, cause a significant reduction in the yield of zootechnic productions (flies, horseflies, mosquitoes).

As this technology is based on a product which must be ingested by the target organism, it can only be applied for fighting animals which chew the tissues of plants or which feed on scraps or food products. In the case of phytophages, the product can be easily sprayed on the leaves and stem of plants, whereas in the case of animals infesting household environments, the product can be mixed or sprayed on appropriate baits.

The technology in question however cannot be applied for fighting plant-sucking insects which, by perforating the vegetable tissue to have access to its lymph, do not come into contact with the product sprayed on the surface.

The advantages which can be associated with the technology, object of the present invention, can be summarized as follows:

a) the selectivity, which can harm a certain animal pest, but not other organisms which make use of the same food (human beings or animals) or which accidentally come into contact with the product (benevolent animals which live in the same environment);
b) food safety, for the same reason as above, but also due to the fact that an RNA molecule can be easily degraded and does not remain for a long period in the environment;
c) potential absence of allergenic risk, as proteins or chemical substances which can be toxic or with antigenic properties are not used;
d) facility of use and application of the product which can be easily sprayed onto plants as if it were a simple chemical product.

An object of the present invention therefore relates to transgenic microorganisms transformed with at least one expression vector comprising a cDNA sequence encoding a GPCR receptor which is vital for a phytophagous animal or pest, or a portion thereof, said receptor being selected from the group consisting of allatostatin, diuretic hormone, octopamine/tyramine, PBAN peptide, adipokinetic hormone, neuropeptide, dopamine, serotonin, gonadotropin receptors, as well as olfactory or rhodopsin-like receptors, said microorganisms being characterized by the fact that they have been inactivated.

The term microorganism in the present invention comprises prokaryotic and eukaryotic cells selected from the groups which consists of bacteria, yeasts, fungi, protozoa, insect cells, mammalian cells or plant cells.

According to a particularly preferred embodiment of the invention, said microorganism is E. coli. According to alternative embodiments, said microorganism is a yeast, preferably selected from Saccharomyces and Pichia.

In a preferred embodiment, said microorganisms are transformed with a cDNA encoding a GPCR receptor which is vital for Spodoptera littoralis having a nucleotide sequence selected from the group consisting in allatostatin receptor C AlstCR (SEQ ID NO:1 encoding SEQ ID NO:2), allostatin A receptor, AlstAR (sequence FJ374689 encoding the sequence ACJ06649; SEQ ID NO: 9-SEQ ID NO:10), diuretic hormone receptor, DHR (sequence FJ374690 encoding the sequence ACJ06650; SEQ ID NO:11-SEQ ID NO:12), adipokinetic hormone receptor, AKHR (SEQ ID NO:3 encoding SEQ ID NO:4), hormone receptor LGR1 (sequence FJ374692 encoding the sequence ACJ06652; SEQ ID NO:13-SEQ ID NO:14), receptor of octopamine/tyramine, Oct/TyrR (sequence FJ374691 encoding the sequence ACJ06651; SEQ ID NO:15-SEQ ID NO:16), olfactory receptor OR83b (sequence FJ374688 encoding the sequence ACJ06648; SEQ ID NO:17-SEQ ID NO:18) and the PBAN, PBAN-R peptide receptor (sequence ABD52277 encoding the sequence DQ407742; SEQ ID NO:19-SEQ ID NO:20).

In a further preferred embodiment, said microorganisms are transformed with a cDNA encoding a GPCR receptor vital for Musca domestica having the nucleotide sequence (SEQ ID NO:21) encoding the receptor of octopamine having SEQ ID NO:22.

Again according to another embodiment of the present invention, said microorganisms are transformed with a cDNA encoding a GPCR receptor vital for Leptinotarsa decemlineata having the nucleotide sequence (SEQ ID NO:23) encoding the receptor of octopamine having SEQ ID NO:24.

A further object of the present invention relates to a method for obtaining transgenic microorganisms containing at least one dsRNA capable of inhibiting the functionality of a GPCR receptor suitable for eliminating the attack of one or more phytophagous organisms or pests, comprising the following phases:

a) isolation of the cDNA nucleotide sequence encoding a receptor coupled with G proteins (GPCR), or portion thereof, which exerts functions of vital importance for the phytophagous organism or pest, selected from the group consisting in receptors for allatostatin, diuretic hormone, octopamine/tyramine, PBAN peptide, adipokinetic hormone, neuropeptide, dopamine, serotonin, gonadotropin, as well as olfactory or rhodopsin-like receptors;
b) construction of an appropriate expression vector comprising the cDNA nucleotide sequence encoding a GPCR receptor or portion thereof, as determined in step a); each expression vector is selected on the basis of the microorganism which is to be used as dsRNA vector, an operation which falls within the knowledge of an expert in the field;
c) transfer of said vector or recombinant plasmid into the cell of a microorganism selected from bacteria, yeasts, fungi, protozoa, or into a eukaryotic cell in culture selected from insect cells, mammalian cells or plant cells for dsRNA expression;
d) growth of the microorganisms or eukaryotic cell culture expressing dsRNA, until dense cultures are obtained;
e) spinning the cell culture to eliminate the culture medium, washing the pellet obtained and lyophilization to eliminate all of the liquid component;
f) inactivation of the microorganisms by heat or by other physical procedures.

According to the present invention, “dense culture” refers in the case of bacteria to cultures with an optical density higher than 0.6 DO, whereas in the case of eukaryotic cells a number of cells higher than 10⁷ per millilitre.

The present invention also relates to transgenic microorganisms containing at least one dsRNA capable of inhibiting the functionality of a GPCR receptor of a phytophage or pest which can be obtained with the preparation method indicated above.

A further object of the present invention relates to the use of the microorganisms as defined above, alone or associated with other strains of microorganisms transformed with a cDNA encoding a GPCR receptor vital for a different phytophage or pest, as pesticides against larvas and/or adult insects.

According to a preferred embodiment, the invention relates to the specific use of microorganisms (i.e. E. coli) transformed with a cDNA encoding a GPCR receptor vital for Spodoptera littoralis having a nucleotide sequence selected from the group consisting in AlstAR (sequence FJ374689 encoding the sequence ACJ06649; SEQ ID NO: 9-SEQ ID NO:10), AlstCR (SEQ ID NO:1-SEQ ID NO:2), DHR (sequence FJ374690 encoding the sequence ACJ06650; SEQ ID NO: 11-SEQ ID NO: 12), AKHR (SEQ ID NO:3-SEQ ID NO:4), LGR1 (sequence FJ374692 encoding the sequence ACJ06652; SEQ ID NO: 13-SEQ ID NO:14), Oct/TyrR (sequence FJ374691 encoding the sequence ACJ06651; SEQ ID NO:15-SEQ ID NO:16), OR83b (sequence FJ374688 encoding the sequence ACJ06648; SEQ ID NO:17-SEQ ID NO:18) and PBANR (sequence ABD52277 encoding the sequence DQ407742; SEQ ID NO:19-SEQ ID NO:20), as a pesticide for the eradication of the lepidopter Spodoptera littoralis.

Alternatively, the invention relates to the specific use of microorganisms (i.e. E. coli) transformed with a cDNA encoding a GPCR receptor vital for Musca domestica having the nucleotide sequence (SEQ ID NO:21) encoding the receptor of octopamine having SEQ ID NO:22, as pesticide for the eradication of the dipteran Musca domestica. According to a further preferred embodiment, the invention relates to the specific use of microorganisms (i.e. E. coli) transformed with a cDNA encoding a GPCR receptor vital for Leptinotarsa decemlineata having the nucleotide sequence (SEQ ID NO:23) encoding the receptor of octopamine having SEQ ID NO:24, as pesticide for the eradication of the coleopteran Leptinotarsa decemlineata.

The invention also relates to a pesticide composition comprising the transgenic microorganisms as defined above as active ingredient at a concentration ranging from 1,000 to 30,000 ppm, in the presence or absence of excipients and/or adjuvants in use in the agricultural field or pesticide formulations.

In an alternative embodiment, said transgenic microorganisms can be different strains of the same or of a different microorganism transformed with cDNA encoding a GPCR receptor vital for different phytophages and/or pests. The method according to the present invention, in fact, envisages the selection of one or more GPCR receptors as RNA interference target, said receptor must be vital for the phytophagous organism or pest against which it should act. In relation to the organism, different types of GPCR receptors can be used as target. Consequently, according to a particularly preferred embodiment of the invention, phases a)-g) of the method according to the invention can be effected using strains of microorganisms each expressing a dsRNA encoding a GPCR receptor or parts of it, vital for the various species of harmful organisms to be struck.

Preferably, the pesticide composition based on microorganisms expressing dsRNAs specific for a GPCR receptor vital for phytophages or pests, is formulated as a spray, powder, suspension of the microorganisms in aqueous solution, lyophilized preparation, paste. In the application against Spodoptera littoralis, for example, the product according to the invention can be formulated in the form of a powder which is dissolved in water and administered to the larvae (i.e. through the leaves of plants on which they normally feed). In applications against Musca domestica, on the other hand, the product can be mixed directly with food-bait or with excrements of farm animals, on which the larvas of the insect feed.

A further object of the present invention relates to a method for controlling and/or eliminating one or more phytophagous organisms in the adult or larval state for the treatment of plants of agronomical (such as for example cereals, solanaceous plants, vines, vegetables), ornamental (such as roses, etc.) environmental (such as conifers, linden trees, poplar trees, etc.) interest, which comprises spraying a pesticide composition as defined above on the leaves or stem of said plants or in the ground. The plants treated with the products deriving from the present invention for inhibiting the functionality of the GPCR receptors of insects can be used for the production of food destined for human beings or for zootechnic, but also for the production of biomass for industrial purposes (paper, fibres, pharmacological substances, biomasses for energy production or basic chemical substances, etc.).

Said phytophagous organisms are preferably selected from the group consisting of lepidopters (i.e. Spodoptera littoralis), dipterans (i.e. Musca domestica), coleopterans (i.e. Leptinotarsa decemlineata), isopterans, thysanopterans, orthopterans, hymenopterans, xylophagous insects, among molluscs gasteropodes and mites. According to preferred embodiments, the herbivorous organism or pest in the adult or larval state can be selected from the following species (grouped according to the damage they cause):

### 1) Insects Harmful for Fruit Plants:

a) LEPIDOPTERS: Prays citri (flower moth), Phyllocnistis citrulla (citrus-leaf miner), Leucoptera malifoliella (pear-leaf blister moth), Phyllonorycter blancardella (pnigalio Schrank), Cydia pomonella (apple worm), Cydia molesta (oriental peach moth), Cossus cossus (red woodworm);
b) COLEOPTERANS: Otiorhynchus cribricollis (otiorhynchus), Anomala spp. (anomala), Melolontha Melolontha (May-bug), Scolytus spp. (scolytus of almond and peach trees), Curculio spp. (curculionides);
c) DIPTERA: Ceratitis capitata (Mediterranean fruit fly), Rhagoletis cerasi (cherry fly);

### 2) Insects Harmful for Olive Trees:

a) LEPIDOPTERS: Prays oleae (olive moth), Zeuzera pyrina (yellow goat moth);
b) COLEOPTERANS: Otiorrhynchus cribricollis (otiorhynchus);
c) DIPTERA: Bactrocera (=Dacus) oleae (olive moth);
d) ISOPTERANS: Kalotermes flavicollis (yellow neck kalotermes);

### 3) Insects Harmful for Vines:

a) LEPIDOPTERS: Lobesia botrana (vine moth);
b) COLEOPTERANS: Anomala vitis (Anomala);
c) DIPTERA: Ceratitis capitata (Mediterranean fruit fly) THYSANOPTERA: Frankliniella occidentalis (american thunderbug);

### 4) Insects Harmful for Cereals:

a) ORTHOPTERANS: Dociostaurus maroccanus (cross locust);
b) THYSANOPTERANS: Limothrips cerealium (wheat thrips),
c) LEPIDOPTERS: Ostrinia nubilalis (European corn borer), Sesamia spp (corn and sorghum sesamia), Helicoverpa zea (cotton worm);
d) COLEOPTERANS: Agriotes lineatus (elater);
e) DIPTERA: Mayetiola spp (wheat and oat gall-midge), Tipula oleracea (crane-fly);

### 5) Insects Harmful for Herbaceous Plants and Greens:

a) ORTHOPTERANS: Dociostaurus maroccanus (Cruciate locust), Locusta migratoria (locust), Gryllotalpa gryllotalpa (mole cricket), Gryllus spp (spotted country cricket);
b) THYSANOPTERANS: Frankliniella occidentalis (American thunderfly);
c) LEPIDOPTERS: Autographa gamma (plusia gamma), Mamestra brassicae (brassica noctuid), Spodoptera littoralis (spodoptera), Heliotis virescens (tabacco noctuid), Helicoverpa armigera (tomato noctuid), Pieris spp (cabbage butterfly and cabbage white), Tuta absoluta (tomato moth), Helicoverpa zea (cotton worm);
d) COLEOPTERANS: Leptinotarsa decemlineata (Potato-beetle), Agriotes lineatus (click-beetle), Othiorrynchus spp (Othiorrynchus);

### 6) Insects and Mites Infesting Domestic Environments:

1) ORTHOPTERANS: Blatta orientalis (black-beetle or cockroach), American Periplaneta (red cockroach or American cockroach), German Blattella (grey cockroach), Supella longipalpa, (wood black-beetle);
2) DIPTERAN: Musca domestica (common fly), Fannia canicularis (small house fly), Stomoxys calcitrans (haematophagous horsefly), Tabanus bovinus (gadfly), Sarcofaga carnaria (Sarcophagidae fly), Calliphora spp (blue fly) ande Lucilia spp (green fly); Culex pipiens (common mosquito), Aedes aegypti (tiger Egyptian mosquito), Aedes albopictus (tiger mosquito), Anopheles gambie (malaria anopheles);
3) HYMENOPTERA: Monomorium pharaonis (pharaonis ant), Iridomyrmex humilis (Argentinean ant), Lasius niger (black ant), Pheidole pallidula (red ant), Crematogaster scutellaris (bulldog ant);
4) LEPIDOPTERS and COLEOPTERANS, which feed on food products: Plodia interpunctella (food moth), Lasioderma serricorne (tobacco woodworm), Stegobium paniceum (bread woodworm), Sitophilus oryzae (rise-weevil), Tribolium spp (caltrops); or which feed on fabrics (wool, silk, cotton): Tineola bisselliella (tineid), Anthrenus spp and Attagenus spp (carpet coleopterans).
5) XYLOPHAGOUS INSECTS, which perforate and feed on furniture wood, works of art, beams and various structures: Hylotrupes bajulus (house-serow), Anobium punctatum (woodworm), Kalotermes flavicollis and, above all, Reticulitermes lucifugus (white ants);
6) MITES RESPONSIBLE FOR ALLERGIES: Dermatophagoides spp and Glycyphagus domesticus (powder mite).
7) PHYTOPHAGOUS MOLLUSCS: Helix spp., Cepaea spp (snails); Arion spp and Deroceras spp (slugs).

According to a preferred embodiment in which said phytophagous organism is Spodoptera littoralis, the nucleotide sequence of the cDNA encoding a GPCR receptor or a portion thereof, is selected from the group which consists in AlstAR (sequence FJ374689 encoding the sequence ACJ06649; SEQ ID NO: 9), AlstCR (SEQ ID NO:1), DHR (sequence FJ374690 encoding the sequence ACJ06650; SEQ ID NO: 11), AKHR (SEQ ID NO:3), LGR1 (sequence FJ374692 encoding the sequence ACJ06652; SEQ ID NO: 13), Oct/TyrR (sequence FJ374691 encoding the sequence ACJ06651; SEQ ID NO: 15), OR83b (sequence FJ374688 encoding the sequence ACJ06648; SEQ ID NO:17) and PBANR (sequence ABD52277 encoding the sequence DQ407742; SEQ ID NO:19).

With respect to other species of lepidopters, such as for example Heliotis virescens, the authors have found that the GPCR receptors homologous to those of allatostatin C and the diuretic hormone of Spodoptera could represent the election targets for this type of herbivorous insect.

In a further preferred embodiment in which said phytophagous organism is Musca domestica, the cDNA nucleotide sequence encoding a GPCR receptor or a portion thereof has the nucleotide sequence (SEQ ID NO:21) encoding the receptor of octopamine having SEQ ID NO:22. According to a further embodiment of the present invention in which said phytophagous organism is Leptinotarsa decemlineata, the cDNA nucleotide sequence encoding a GPCR receptor or a portion thereof has the nucleotide sequence (SEQ ID NO:23) encoding the receptor of octopamine having SEQ ID NO:24.

Furthermore, the technology in question can also be used for protecting products deriving from the agrarian or food industry. The stored crops, awaiting processing or sale, can undergo the attack of saprophyte organisms. Food products stored in non-cooled places can also be protected from insects and other arthropods with the use of the technology in question. Finally, the technology can be applied in house-holding and veterinary environments against all harmful organisms which infest and can be a threat to the health of human beings, products of human beings and domestic animals.

Finally, the invention relates to a method for the control and/or elimination of pests in the adult or larval state from an environment comprising the application of a pesticide composition in powder form, in solution or by spraying on a surface selected from wood, fabrics or soil, water well, excrements of domestic animals. Said pests are preferably selected from the group which consists of flies, cockroaches, ants, mosquitoes, horse-flies, mites, termites, longhorn-beetles, cloth-moths.

According to a preferred embodiment of the methods for controlling phytophagous organisms and pests of the present invention, one or more microorganisms containing dsRNA can be produced and used for protecting the plant against one or more species of insects or molluscs contemporaneously, or for protecting environments from infestations of harmful organisms. In this way, it is possible to obtain products with two or more types of dsRNA and thus act against various harmful species which co-exist in the same environment. A use of this type of product on plants, for example, would provide protection from the attack of both phytophagous organisms which prevalently feed on leaves, and also organisms which feed on bulbs or roots. Although preserving the specific resistance characteristics to single species of harmful organisms, plants can also be protected from various harmful species, even extremely different from each other, i.e. belonging to groups which are phylogenetically very distant from each other.

The present invention will now be described illustratively but non-limitedly, according to its preferred embodiments with particular reference to the figures of the enclosed drawings, in which:

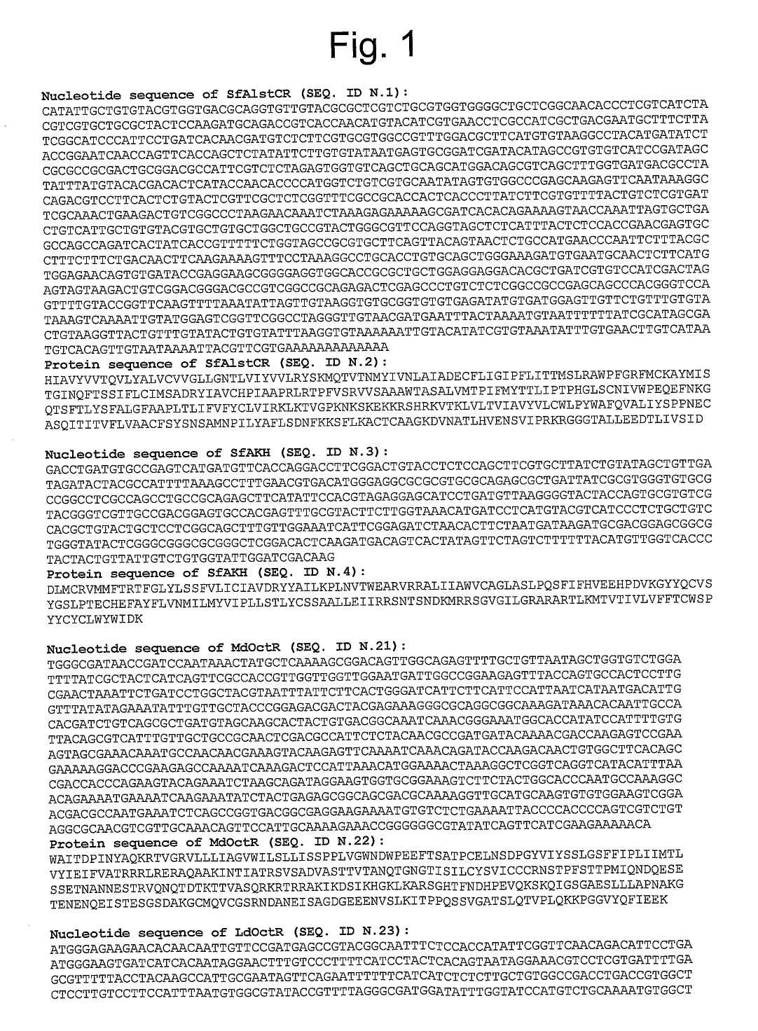

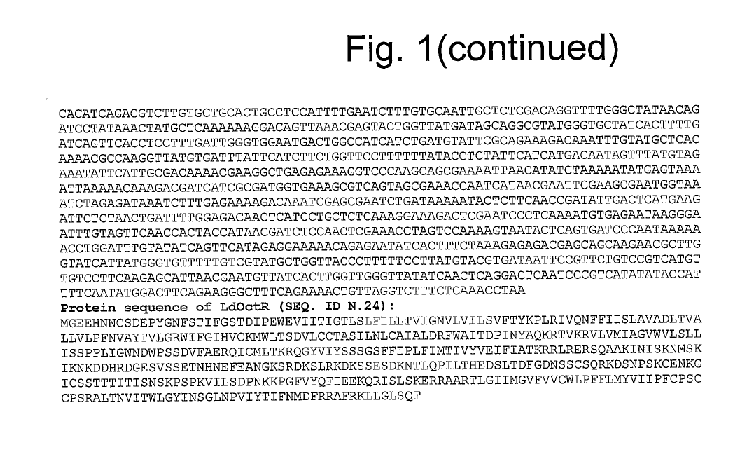

FIG. 1 shows the sequences of the cDNAs encoding the GPCR receptors: AlstCR (SEQ. ID NO:1 and SEQ: ID NO.2) and AKHR (SEQ. ID NO:3 and SEQ. ID NO:4) cloned by S. littoralis;

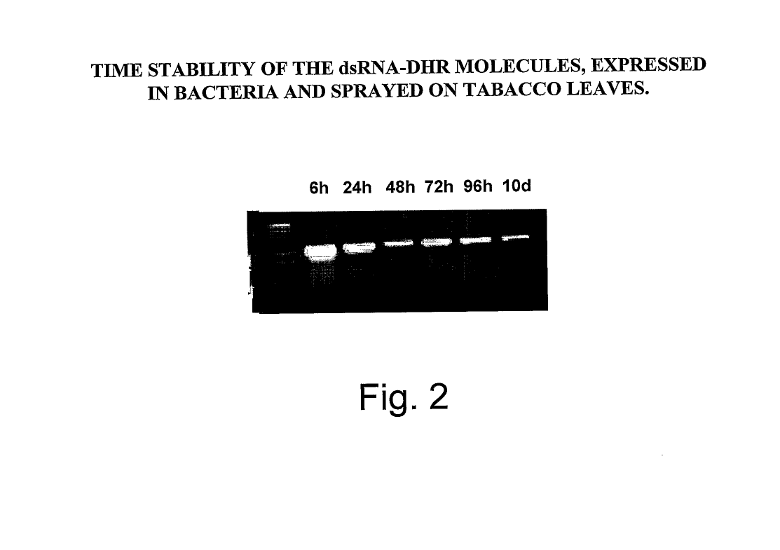

FIG. 2 shows the RT-PCR analysis at different times of E. coli microorganisms expressing the dsRNA (corresponding to the DHR receptor) sprayed on tobacco leaves: the result provides some indications on the stability, and therefore effectiveness of the product with time;

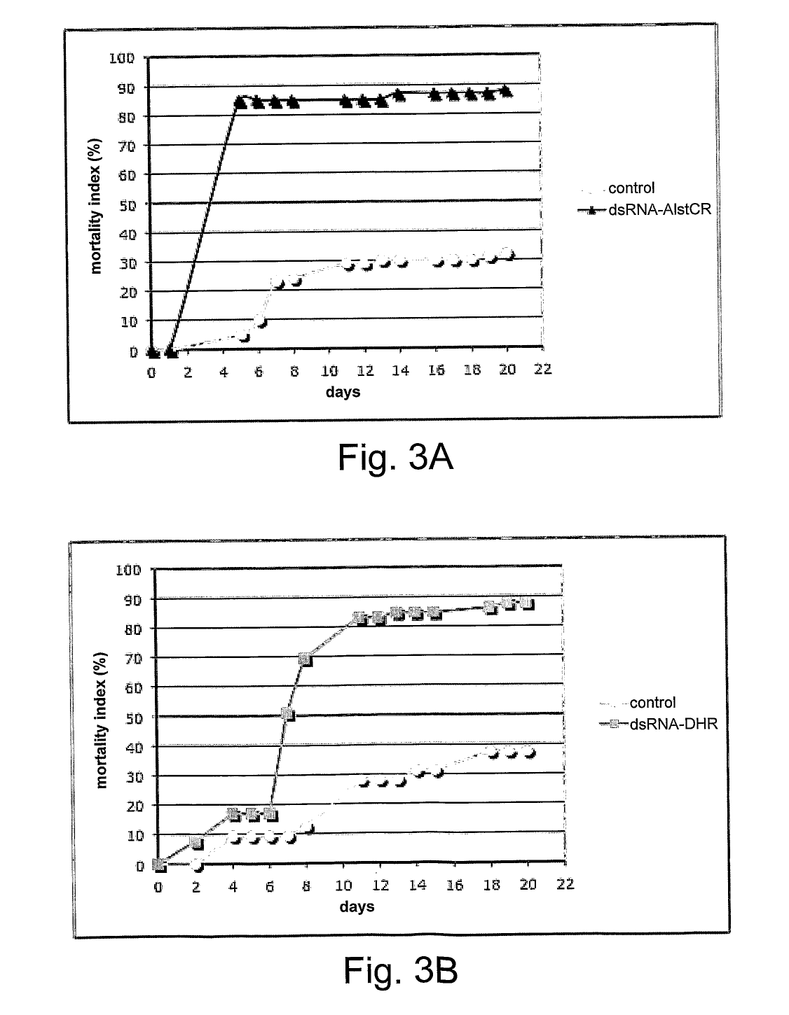

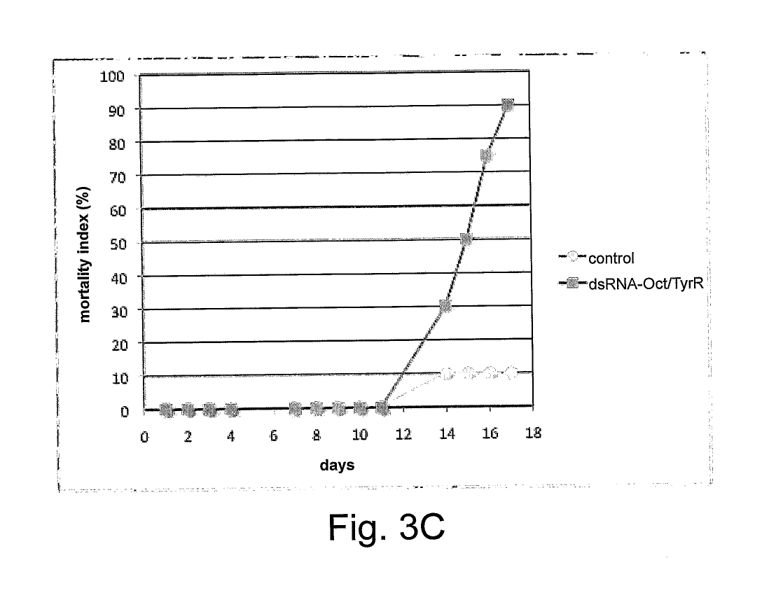

FIG. 3, panels A-C, shows the graphs relating to the mortality percentage of Spodoptera littoralis larvae fed with the artificial diet containing 5,000 ppm of product which specifically inhibits the functionality of the receptors AlstCR, DHR and Oct/TyrR.

For illustrative and non-limiting purposes of the present invention, the production of bacteria of E. coli expressing the dsRNA corresponding to a part of or whole sequence of the GPCR receptors of Spodoptera, AlstCR, DHR and Oct/TyrR, is described hereunder. Once produced, the bacteria were administered to the larvae together with leaves starting from different stages in order to evaluate their toxic effect and measure the mortality rate.

### EXAMPLE 1

### Transformation of E. Coli Bacteria with the Plasmid Containing the GPCR Gene of Spodoptera Littoralis

### Materials and Methods

### Selection of the GPCR Target Receptors

The receptors of S. littoralis selected as target of the present embodiment of the invention are:

1. AlstCR (putative receptor of allatostatin C): this GPCR is of fundamental importance as it binds allatostatins, neuropeptides whose main function is to inhibit the synthesis of the juvenile hormone on the part of the “corpus allatum”. Allatostatins are not only involved in the metamorphosis process and ecdysis (Weaver et al., 1994), but are also important for regulating smooth muscle contractions of the intestines and in the development and functionality of the ovaries in adults (Aguilar et al., 2003; Meyering-Vos et al., 2006);
2. DHR (receptor of the diuretic hormone DH): important for regulating the diuresis (Johnson et al., 2004) and the water balance of the whole body of the insect, during both the larval and adult stage;
3. PBAN-R (receptor of the activation neuropeptide of the biosynthesis of pheromones, PBAN): it binds a small peptide which promotes the synthesis and the release of sexual pheromones (Rafaeli et al., 2007):
4. LGR1 Leucine-Rich Repeat-Containing G Protein-Coupled Receptor): belongs to the group of G protein-coupled receptors containing Leucine-rich repeats (LGR) and exerts a fundamental role in reproduction (Nishi et al., 2000);
5. Oct/TyrR (putative receptor of octopamine and tyramine): it is capable of interacting with neuro-transmitters such as octopamine and tyramine and is therefore important in neuromodulation process in sensorial and secretory organs (Farooqui, 2007);
6. OR83b (receptor of the family of olfactory receptors): unlike other members of the family of olfactory receptors (OR), which are only expressed in small sub-populations of sensorial olfactory neurons, OR83b is expressed in almost all neurons of the feeler (Neuhaus et al., 2005). Rather than having a direct role in the olfactory function, it interacts with the conventional OR members and is essential for their diffusion from the cell bodies to the sensorial cilia where the interaction with the odorant molecules takes place. For the general role it exerts in the olfactory system, OR83b is certainly of fundamental importance for the perception of odours and therefore for the nutrition of the insect.

For effecting the RNAi experiments, all the sequences encoding the GPCR were amplified by the cDNA of Spodoptera with specific primers, with the exception of the genes encoding AlstCR (SEQ. ID NO:1) and AKHR (SEQ. ID NO:3) which were previously cloned by 5′ and 3′ RACE-PCR using degenerated primers.

### Isolation and Cloning of the Selected Receptors.

The sequences encoding the receptors AlstCR and AKHR were isolated using the cDNA retro-transcribed from the total RNA extracted from S. littoralis, using degenerated primers deriving from comparison with regions preserved among these genes in the various insect species.

The following degenerated primers were used for the gene of AlstCR:

1. 1Fw:
(SEQ ID NO: 5)
GA(AG)TG(TC)TT(TC)(TC)T(AGCT)AT(ATC)GG(ATGC);
2. 1Rv:
(SEQ ID NO: 6)
(ATGC)GC(AG)CA(ATGC)GT(AG)CA(ATGC)GC(TC)TT.

The following degenerated primers were used for the gene of AKHR:

3. 1Fw:
(SEQ ID NO: 7)
GACCTGATGTGC(AC)G(AC)(AG)TCATG
4. 1Rv:
(SEQ ID NO: 8)
GTC(AG)ATCCA(AG)TACCACAG(AG)CA.

The PCR products obtained (corresponding to portions of sequence of these receptors) were sequenced and used for designing specific primers for the experiments of 5′ and 3′ RACE. The sequences isolated were cloned in the plasmid pCR® 2.1-TOPO® (Invitrogen) and sequenced (FIG. 1).

For the other receptors (DHR, PBANR, LGR1, Oct/TyrR, OR83b) on the basis of the expression data available in literature, the sequences were isolated using different tissues and growth stages of the insect. In particular, the gene encoding the receptor LGR was amplified from eggs, whereas DHR from V-stage larvas; the genes encoding the receptors Oct/TyrR and OR83b were amplified by RNA extracted from the head of individual adults; V-stage larvas, on the other hand, were used for PBAN-R.

Once cloned in the vector pCR® 2.1-TOPO®, all the genes encoding the receptors were transferred by digestion into the vector L4440. For the genes of AlstCR and DHR, the restriction enzymes KpnI and XhoI were used; for that of PBANR, EcoRI was used, whereas for those of LGR, Oct/TyrR and OR83b, SacI and XhoI were used. The ligation reactions between the genes of the receptors (200 ng) and the vector (300 ng) were effected in a volume of 20 ul at 16° C. using 20 units of T4 DNA ligase (BioLabs). The vector L4440 is a modified version of the plasmid pBluescript containing at both ends of the polilinker the promoter for T7 RNA polymerase, capable of transcribing the gene in both directions. In this way, two transcripts are produced, one “sense” the other “antisense”, which once coupled, form a single dsRNA.

### Transformation and Growth of the Bacterial Cultures:

50 ng of recombinant plasmid, each containing the sequence of the receptor selected or part of it, were transformed into cells of E. coli belonging to the strain HT115 (DE3). The cells were kept in ice for 30 minutes. The thermal shock was effected by keeping the cells for 1 minute at 37° C. and then 2 minutes in ice. 1 ml of YEP was added to the cells. After 1 hour of growth at 37° C., the cells were plated on YEP-agar containing tetracycline 12.5 ug/ul and ampicillin 100 ug/ul. The plate was left overnight at 37° C. A transformed colony was inoculated in YEP containing tetracycline 12.5 ug/ul and ampicillin 100 ug/ul and was grown for 18-20 hours. The following day the cells were diluted in 500 ml of YEP containing antibiotics and grown until reaching an OD600 (optical density measured at 600 nm) equal to 0.4. The specific gene expression was induced with IPTG 0.4 mM. After induction, the cells were left to grow all night at 37° C. The cells were centrifuged at 6,000 rpm for 15 minutes. The pellet obtained was frozen and then lyophilized. After the lyophilization, the pellet obtained was boiled for about 15 minutes to deactivate the bacteria. For the tests on insects, the pellet containing the dsRNA was resuspended in water and used at the selected concentration.

Tests with larvae of S. littoralis:

Larvae of S. littoralis of different stages were used for the biological tests. A number varying from 70 to 200 larvae were used for each experiment. After the eggs had hatched, the larvae were first kept together in a single container in the presence of food, then isolated starting from stage I, II or III. Each larva was placed in a container having a diameter of 6 cm into which about 3 g of food containing from 5,000 to 15,000 ppm of bacterial lyophlized product, was introduced daily. Every 2 days, the food was substituted with fresh food, whereas every day the number of dead and alive larvae were counted to calculate the mortality percentage of the populations with time. The food used for the larvae, “pabulum”, had the following composition: for each 1.14 l of water, 33.75 g of agar, 47.27 g of wheat germs, 67.5 g of beer yeast, 189 g corn flour, 6.75 g of ascorbic acid, 1.7 g benzoic acid and 1.35 g of nipagin, are added.

### Results

Once the E. coli bacteria had been transformed with the plasmid L4440 for the expression of the dsRNA, they were lyophilized and used for verifying the effect of the dsRNA contained therein on the vitality of the larvae of S. littoralis.

Before administering the dsRNA contained in the bacteria to the larvas, its stability with time was verified to ensure that the larvas are fed each day with a product containing dsRNA molecules which are intact and consequently functioning for the gene silencing. Bacteria containing dsRNA molecules corresponding to the DHR receptor, suitably deactivated, were therefore sprayed onto tobacco leaves removed from the plant at a concentration of 5,000 ppm. The dsRNA was re-isolated at intervals of 24 hours by the extraction of RNA and amplified with specific primers which allow the whole of its gene sequence to be visualized. As shown in FIG. 2, even after 10 days, the presence of dsRNA molecules can be detected on the leaf, even if the quantity is much lower than that observed only a few hours after the treatment. This result confirms that the product, object of this invention can be used, as it is stable with time and moreover does not persist for excessively long times.

Once the stability of the dsRNA had been verified, a series of experiments were prepared, in which larvae starting from stage I were fed with “pabulum” mixed with 5,000 ppm of bacteria containing dsRNA corresponding to the gene of AlstCR. In these experiments, about 200 larvae were used, fed with bacteria expressing dsRNA right from the first stage after the hatching of the eggs, and compared with the same number of control larvae, fed however with a diet containing bacteria not expressing dsRNA. FIG. 3 (graph A) shows the graph of the trend of the mortality rate of a population of control larvae with respect to a population fed with bacteria expressing dsRNA. Even after a few days, the effect of the dsRNA on the vitality of the larvae is evident: after 20 days (shortly before the metamorphosis), the mortality rate of the population of larvae fed with dsRNA reached 90%, whereas that of the control larvae was about 30%. It is consequently evident that the population of larvae fed with the diet containing the product of the invention shows a much higher mortality percentage with respect to the larvae fed with the control diet.

In a second series of experiments, mortality rates were measured in populations of larvae (each of 70 individuals) fed starting from stage III with 10,000 ppm of bacteria containing dsRNA corresponding to the gene of the DHR receptor, and again compared with control larvae fed with a diet containing bacteria without dsRNA. FIG. 3 (graph B) shows the results of this experiment, which, also in this case, clearly indicate the significant effect of dsRNA on the vitality of the larvae: after about 20 days, the mortality rate is about 90% in the population treated, against 40% of the control population.

Finally, a third experiment was performed, also testing the effect of dsRNA corresponding to the gene of the receptor of octopamine/tyramine (Oct/TyrR) on larvae starting from stage III, analogously to what was done for the DHR receptor. The results, shown in FIG. 3 (graph C) indicate that also for the Oct/TyrR receptor, the mortality rate of the larvae fed with dsRNA reaches 90% after 17 days, against only 10% of the control population fed with bacteria not expressing dsRNA.

All three experiments described, effected on three different types of dsRNA (i.e. corresponding to three different GPCR targets), clearly suggest that the technology in question, which the applicant intends to protect, is valid for the control of phytophagous organisms and can also be extended to all pests harmful both for agriculture and for housing, for human or animal use.

### EXAMPLE 2

### Transformation of E. Coli Bacteria with the Plasmid Containing the Gene of the Octopamine Receptor of Musca domestica

### Materials and Methods

### Selection of the GPCR Target Receptor

The receptor of M. domestica selected as target of the present embodiment of the invention is that of octopamine (MdOctR). It is in fact of fundamental importance in insects, as it is capable of interacting with neuro-transmitters such as octopamine and tyramine during all neuromodulation processes in sensorial and secretory organs (Farooqui, 2007).

### Isolation and Cloning of the Receptor Selected

Part of the sequence encoding the receptor MdOctR was isolated using the cDNA retro-transcribed from the total RNA extracted from heads of adult individuals of M. domestica, using degenerated primers deriving from a comparison with regions preserved among these genes in the various insect species, as also in the case of the isolation of the sequences of S. littoralis. The degenerated primers used are the following:

1. 1Fw:
(SEQ ID NO: 25)
TGGGC(ATCG)AT(ATC)AC(ATCG)GA(TC)CC(ATCG)AT(ATC)AA
(TC);
2. 1Rv:
(SEQ ID NO: 26)
CA(ATCG)AC(ATCG)AC(AG)AA(ATCG)AC(ATCG)CCCAT(ATG)
AT.

The PCR product obtained (corresponding to a portion of sequence of the receptor) was cloned in the plasmid pCR® 2.1-TOPO® (Invitrogen) and sequenced (FIG. 1).

Once it had been cloned in the vector pCR® 2.1-TOPO®, the gene was transferred by digestion into the vector L4440, as done for the genes of S. littoralis.

### Transformation and Growth of the Bacterial Culture

50 ng of recombinant plasmid, containing part of the sequence of the receptor MdOctR, was transformed into cells of E. coli belonging to the strain HT115 (DE3). The whole procedure used, from the transformation of the bacterial cells to the production of dsRNA, is identical to that adopted for the genes of S. littoralis, already amply described above.

Tests with Larvae of M. domestica:

Three day old larvae of M. domestica were used for the biological tests. 15 larvae were used for each experiment, which were placed in 50 ml containers containing about 10 g of artificial diet mixed with 2 ml of product dissolved in water, so as to have a final concentration of the product of about 3,000-5,000 ppm. At the end of the larval cycle, which lasts 6 days, the larvae were analyzed to calculate the mortality rate. The food used for the larvae has the following composition: 100 g of wheat bran and 20 g of powder milk are added to each 100 ml of demineralized water.

### Results

Once the stability of the dsRNA had been verified, a series of experiments were prepared, in which the larvae, starting from the third day after the hatching of the eggs, were fed with an artificial diet mixed with 3,000-5,000 ppm of bacteria containing dsRNA corresponding to the gene of MdOctR. In these experiments, 15 larvae were used, fed with bacteria expressing dsRNA, and compared with the same number of control larvae, fed however with a diet containing bacteria not expressing dsRNA. The average mortality rate was calculated in the populations of larvae fed with bacteria expressing dsRNA and in those fed with bacteria not expressing dsRNA, 6 days after the hatching of the eggs, i.e. at the end of the larval cycle and prior to the pupal stage. The average mortality rate of the populations of larvae fed with dsRNA reached 34%, whereas that of the control larvae was only 13%. It is therefore evident that the larvae fed with the diet containing the product of the invention show a much high mortality percentage with respect to larvae fed with the control diet.

### EXAMPLE 3

### Transformation of E. coli Bacteria with the Plasmid Containing the Gene of the Octopamine Receptor of Leptinotarsa decemlineata

### Materials and Methods

### Isolation and Cloning of the Receptor Selected

Also for Leptinotarsa decemlineata the receptor selected as target of the present embodiment of the invention is that of octopamine (LdOctR).

Part of the sequence encoding the receptor LdOctR was isolated using the cDNA retro-transcribed from the total RNA extracted from heads of L. decemlineata, in the adult stage, using degenerated primers deriving from a comparison with regions preserved among these genes in the various insect species, as also in the case of the isolation of the sequences of S. littoralis. The degenerated primers used for this cloning are the same as those used for the cloning of the MdOctR receptor of M. domestica (SEQ. ID N. 25 and 26).

The PCR product obtained (corresponding to a portion of sequence of this receptor) was sequenced and used for designing specific primers for the experiments of 5′ and 3′ RACE. The sequence isolated, corresponding to the whole gene of the receptor, was cloned in the plasmid pCR® 2.1-TOPO® (Invitrogen) and sequenced (FIG. 1).

Once it had been cloned in the vector pCR® 2.1-TOPO®, the gene was transferred by digestion into the vector L4440, as effected for the genes of S. littoralis.

### Transformation and Growth of the Bacterial Culture:

50 ng of recombinant plasmid, containing the whole sequence of the receptor LdOctR, were transformed into cells of E. coli belonging to the strain HT115 (DE3). The whole procedure used, from the transformation of the bacterial cells to the production of dsRNA, is identical to that adopted for the genes of S. littoralis and M. domestica, already amply described above.

Tests with Larvae of L. decemlineata:

Larvae of L. decemlineata were used for the biological tests, starting from the first stage. About 20 larvae deriving from the hatching of eggs situated on leaves of potato plants were used for each experiment. After the hatching of the eggs, the larvae were fed with leaves of the control plants (treated with bacteria not containing dsRNA) and plants sprayed with an aqueous solution of the product (bacteria containing dsRNA) at a concentration of 15,000 ppm. The larvae were analyzed during the first and second stage, as far as the pupal stage, to calculate the mortality rate.

### Results

Once the stability of the dsRNA had been verified, a series of experiments were prepared to calculate the effect of the product on the larvae of Leptinotarsa, during the various growth stages. Populations of 20 larvae were used in these experiments, fed with bacteria expressing dsRNA right from the first stage after the hatching of the eggs, and compared with the same number of control larvae, fed instead with a diet containing bacteria not expressing dsRNA. Although the experiments are still underway, some preliminary data indicate that also for Leptinotarsa the mortality rate of the larvae fed with the product of the invention is higher with respect to that of the larvae fed with the control diet.

### BIBLIOGRAPHY

Aguilar R, Maestro J L, Vilaplana L, Pascual N, Piulachs M D, Belles X (2003). Regul Pept. Oct. 15; 115(3):171-7.
Baum J A, Bogaert T, Clinton W, Heck G H, Feldman P, Ilagan O, Johnson S, Plaetinck G, Munyikwa T, Pleau M, vaughn TY and Roberts J (2007). Nature Biotech, 25 (11): 1322-1326.
Brantl, S. (2002) Biochimica et Biophysica Acta. 1575:15-25.
Farooqui T (2007). Neurochem Res. 32: 1511-1529.
Hamilton, A. J. and Baulcombe, D. C. (1999). Science 286: 950-952.
Hammond, S. M. Boettcher, S., Caudy, A., Kobayashi, R. and Hannon, G. (2001). Science 293: 1146-1150.
Hill S. J. (2006). Br J. Pharmacol. 147 (S1): S27-S37.
Johnson E C, Bohn L M and Taghert P H (2004). Journal of Exper Biol, 207: 743-748.
Meyering-Vos M, Merz S, Sertkol M, Hoffmann K H (2006). Insect Biochem Mol. Biol. June; 36(6):492-504.
Neuhaus E M, Gisselmann G, Zhang W, Dooley R, Stortkuhl K Hatt H (2005). Nature Neurosc, 8 (1): 15-17.
Nishi S, Hsu S Y, Zell K and Hsuch J W (2000). Endocrinology, 141 (11): 4081-4090.
Rafaeli A, Bober R, Becker L, Choi M Y, Fuerst E J and Jurenka R (2007). Insect Molec Biol, 16 (3): 287-293.
Tomoyasu, Y. and Denell, R. E. (2004). Dev. Genes Evol. 214:575-578.
Weaver R J, Freeman Z A, Pickering M G, Edwards J P. (1994). Comp. Biochem. Physiol. C 107:119-27.

1. Transgenic microorganisms transformed with at least one expression vector comprising a cDNA sequence encoding a GPCR receptor which is vital for a phytophagous organism or pest, or a portion thereof, said receptor being selected from the group consisting of allatostatin, diuretic hormone, octopamine/tyramine, PBAN peptide, adipokinetic hormone, neuropeptide, dopamine, serotonin, gonadotropin receptors, as well as olfactory or rhodopsin-like receptors, said microorganisms being further characterized in that they have been inactivated.

2. The microorganisms as defined according to claim 1, selected from the group consisting in bacteria, yeasts, fungi, protozoa, insect cells, mammalian cells or plant cells.

3. The microorganism according to claim 1, selected from E. coli, Saccharomyces or Pichia.

4. The microorganisms according to claim 1, transformed with a cDNA sequence encoding a GPCR receptor which is vital for Spodoptera littoralis and having a nucleotide sequence selected from the group consisting in allatostatin C receptor AlstCR (SEQ ID NO:1), allostatin A receptor, AlstAR (SEQ ID NO: 9), diuretic hormone receptor, DHR (SEQ ID NO: 11), adipokinetic hormone receptor, AKHR (SEQ ID NO:3), hormone receptor LGR1 (SEQ ID NO: 13), receptor of octopamine/tyramine, Oct/TyrR (SEQ ID NO: 15), olfactory receptor OR83b (SEQ ID NO: 17) and the PBAN peptide receptor (SEQ ID NO: 19).

5. The microorganisms according to claim 1, transformed with a cDNA encoding a GPCR receptor which is vital for Musca domestica having the nucleotide sequence (SEQ ID NO:21) encoding the receptor of octopamine having SEQ ID NO:22.

6. The microorganisms according to claim 1, transformed with a cDNA encoding a GPCR receptor which is vital for Leptinotarsa decemlineata having the nucleotide sequence (SEQ ID NO:23) encoding the receptor of octopamine having SEQ ID NO:24.

7. A method for obtaining transgenic microorganisms suitable for eliminating the attack of one or more phytophagous organisms or pests, containing at least one dsRNA capable of inhibiting the functionality of a GPCR receptor, comprising the following steps:
a) isolation of the cDNA nucleotide sequence encoding a receptor coupled with protein G (GPCR) or a portion thereof, which exerts functions of vital importance for the phytophagous organism or pest of interest, said receptor being selected from the group consisting in allatostatin, diuretic hormone, octopamine/tyramine, PBAN peptide, adipokinetic hormone, neuropeptide, dopamine, serotonin, gonadotropin receptors, as well as olfactory or rhodopsin-like receptors;
b) construction of an expression vector or recombinant plasmid comprising the cDNA nucleotide sequence encoding a GPCR receptor or portion thereof, as determined in step a);
c) transfer of said vector or recombinant plasmid into the cells of a microorganism selected from bacteria, yeasts, fungi, protozoa, or into a eukaryotic cell in culture selected from insect cells, mammalian cells or plant cells for dsRNA expression;
d) growth of the culture of the microorganism or eukariotic cells expressing dsRNA, until dense cultures are obtained;
e) centrifugation of the cell culture to remove the culture medium, washing of the pellet obtained and lyophilization to eliminate all the liquid component;
f) inactivation of the microorganisms by heat or by other physical procedures.

8. Inactivated transgenic microorganisms containing at least one dsRNA capable of inhibiting the functionality of a GPCR receptor of a phytophagous organism or pest, obtainable by the method according to claim 7.

9. Use of the inactivated transgenic microorganism, as defined in claim 1, alone or in association with other strains of microorganisms transformed with a cDNA sequence encoding a GPCR receptor which is vital for a different phytophagous organism or pest, as pesticides against larvae and/or adult insects.

10. Use of the inactivated transgenic microorganisms, as defined according to claim 4, as pesticide for the eradication of the phytophagous organism Spodoptera littoralis.

11. Use of the inactivated transgenic microorganisms, as defined according to claim 5, as pesticide for the eradication of the dipteran Musca domestica.

12. Use of the inactivated transgenic microorganisms, as defined according to claim 6, as pesticide for the eradication of the coleopteran Leptinotarsa decemlineata.

13. A pesticide composition comprising the inactivated transgenic microorganisms, as defined in claim 1, as active ingredient at a concentration ranging from 1,000 to 30,000 ppm in aqueous solution, optionally in the presence of excipients and/or enhancers used in the agricultural field or in pesticide formulations.

14. The pesticide composition according to claim 13, wherein said microorganisms can be different strains belonging to the same species, each strain transformed with a cDNA encoding a GPCR receptor which is vital for different phytophagous organisms and/or pests in the adult and/or larval stage.

15. The pesticide composition according to claim 13, formulated as a spray, powder, suspension of the microorganisms in aqueous solution, lyophilized preparation, paste.

16. A method for the control and/or elimination of one or more phytophagous organisms or pests in the adult or larval stage, for the treatment of plants of agronomical, ornamental and environmental interest, comprising spraying the pesticide composition, as defined according to claim 13, on the leaves or on the stem of said plants or in the ground.

17. The method according to claim 16, wherein said phytophagous organisms or pests are selected from the group consisting of Lepidoptera, Diptera, Coleoptera, Isoptera, Tisanoptera, Orthoptera, Hymenoptera, xylophagous insects, Gastropoda molluscs and mites.

18. A method for the control and/or elimination of pests in the adult or larval stage from an environment, comprising the application of a pesticide composition as a powder, solution or by spraying on a surface selected from wood, cloth, soil, water ponds, animal excrements.

19. The method according to claim 18, wherein said pests are selected from the group consisting of flies, cockroaches, ants, mosquitoes, horse-flies, mites, termites, longhorn-beetles, cloth-moths.

20. The method according to claim 16, wherein said microorganisms can be used alone or in a combination with other strains of microorganisms transformed with a cDNA encoding a GPCR receptor which is vital for a different phytophagous organism or pest in the adult and/or larval stage.
